(19) United States
(12) Patent Application Publication
(10) Pub. No.: US 2023/0172919 A1
(43) Pub. Date: Jun. 8, 2023

(54) AN INSULIN SENSITIZER FOR USE IN THE PREVENTION AND/OR TREATMENT OF THE DAMAGES CAUSED BY RETINAL DETACHMENT

(71) Applicants: SORBONNE UNIVERSITE, Paris (FR); INSERM (INSTITUT NATIONAL DE LA SANTÉ ET DE LA RECHERCHE MÉDICALE), Cedex 13 Paris (FR); CENTRE NATIONAL DE LA RECHERCHE SCIENTIFIQUE, Paris (FR)

(72) Inventors: Florian SENNLAUB, Paris (FR); Jean-Baptiste CONART, Nancy (FR); José-Alain SAHEL, Paris (FR)

(73) Assignees: SORBONNE UNIVERSITE, Paris (FR); INSERM (INSTITUT NATIONAL DE LA SANTÉ ET DE LA RECHERCHE MÉDICALE), Cedex 13 Paris (FR); CENTRE NATIONAL DE LA RECHERCHE SCIENTIFIQUE, Paris (FR)

(21) Appl. No.: 17/913,268

(22) PCT Filed: Mar. 26, 2021
(86) PCT No.: PCT/EP2021/058053

§ 371 Date: Sep. 21, 2022

(30) Foreign Application Priority Data
Mar. 26, 2020 (EP) 20166046.1

(51) Int. Cl. A61K 31/4439; A61K 9/00; A61P 27/02; A61K 45/06; A61K 31/155

(52) U.S. Cl. CPC A61K 31/4439; A61K 9/0019; A61K 9/0048; A61K 31/155; A61K 45/06; A61P 27/02

(57) ABSTRACT

An insulin sensitizer for use in the prevention and/or treatment of the damages caused by retinal detachment, and in particular for preventing or treating the loss of vision induced by retinal detachment. Also, a composition including the insulin sensitizer for use in the prevention and/or treatment of the damages caused by retinal detachment and a device including the composition.

### FIELD OF INVENTION

The present invention pertains to the field of ophthalmic compositions. In particular, the invention relates to a composition comprising at least insulin and/or one insulin sensitizer for use for preventing or treating the damages caused by retinal detachment. The present invention further relates to a method for the prevention or treatment of damages induced by retinal detachment, wherein said method comprises a step of intraocular administration of a composition comprising at least one insulin or insulin sensitizer.

### BACKGROUND OF INVENTION

Retinal detachment is a sight-threatening condition with an annual incidence of 10.5 per 100,000 people. Advances in surgical techniques over recent decades have greatly improved the anatomical results with a primary success rate currently up to 80%. However, despite a successful retinal reattachment, visual recovery may still be disappointing, especially in cases involving the macula and this loss of vision is primarily due to photoreceptor cell death. The physical separation of the neurosensory retina from the underlying retinal pigment epithelium (RPE) and choroidal vasculature indeed impairs the oxygen and nutrient supply to the photoreceptor cells, which eventually die mainly through apoptosis. Several pathogenic mechanisms have been identified but accumulating evidence suggests that inflammation plays a key role in the pathogenesis of retinal detachment-induced photoreceptor cell death. Human studies have thus reported elevated levels of cytokines and chemokines in the vitreous of patients with retinal detachment. Furthermore, experimental models have demonstrated that cytokines from infiltrating mononuclear phagocytes (MP) induce the death of photoceptors following retinal detachment.

Although cones represent only 5% of all photoreceptor cells in humans, they are responsible for daylight, high-acuity and color vision. In retinal detachment, a decrease in cone density in eyes that underwent successful surgery and a strong correlation between cone density and visual acuity is observed.

There is a need of an efficient treatment of cone loss in case of retinal detachment.

### SUMMARY

The present invention deals with an insulin sensitizer for use in the prevention and/or treatment of the damages caused by retinal detachment.

In one embodiment, the insulin sensitizer for are for use in the prevention and/or treatment of the damages caused by retinal detachment, wherein the damages caused by retinal detachment is cone cell death.

In another embodiment the insulin sensitizer for use in the prevention and/or treatment of the damages caused by retinal detachment, wherein the damages caused by retinal detachment is loss of vision.

In another embodiment, the invention deals with a composition for use in the prevention and/or treatment of the damages caused by retinal detachment, wherein said composition comprises at least one insulin sensitizer, wherein preferably the damages caused by retinal detachment is cone cell death and/or loss of vision.

According to a more specific aspect, the composition for use in the prevention and/or treatment of the damages caused by retinal detachment is a pharmaceutical composition and further comprising at least one pharmaceutically acceptable excipient.

According to another specific aspect, the composition for use in the prevention and/or treatment of the damages caused by retinal detachment, further comprises at least one therapeutic agent; preferably an anti-inflammatory, anti-fibrotic, anesthetic, anti-proliferative, antiseptic, anti-infective, antioxidant and/or reactive oxygen species (ROS) scavenger agent.

According to another specific aspect, the composition for use in the prevention and/or treatment of the damages caused by retinal detachment, further comprises at least one buffering agent, osmotic agent and/or colorant.

According to another specific aspect, the composition for use in the prevention and/or treatment of the damages caused by retinal detachment, the composition is an injectable solution; preferably an aqueous solution or a hydrogel solution.

According to another specific aspect, the insulin sensitizer or the composition for use in the prevention and/or treatment of the damages caused by retinal detachment, comprises an insulin sensitizer chosen among direct PPAR agonists, selective PPAR gamma modulators and PPAR gamma-sparing compounds. Direct PPARs agonists can be chosen among the non-limitative list comprising thiazolidinediones such as rosigliatazone, pioglitazone, Edaglitazone, GW1929, troglitazone, Ciglitazone, or Troglitazone, selective PPAR gamma modulators can be chosen among the non-limitative list comprising INT131, CMHX008, nTZDpa, S26948 or Pseudoginsenoside F11, PPAR gamma-sparing compounds can be chosen among the non-limitative list comprising ligand/modulator of the mTOT, agents target to the downstream effectors of PPAR gamma, stimulation of HSP/NOS (BGP-15), 11 beta-HSD1 inhibitor (INCB13739), AMPK activators, biguadine (metformin) or MSDC-0602K, chloroquines (CQs).

In another embodiment of the invention, the insulin sensitizer or the composition for use in the prevention and/or treatment of the damages caused by retinal detachment is to be administered to a subject at risk of retinal detachment, preferably before retinal detachment.

In another embodiment of the invention, the insulin sensitizer or the composition for use in the prevention and/or treatment of the damages caused by retinal detachment is to be administered to a subject after initiation of retinal detachment.

In another embodiment of the invention, the insulin sensitizer or the composition for use in the prevention and/or treatment of the damages caused by retinal detachment is to be administered as long as there are functional cone photoreceptors.

In another embodiment of the invention, the insulin sensitizer or the composition for use in the prevention and/or treatment of the damages caused by retinal detachment is to be administered by direct retinal, subretinal or intravitreal injection.

In another embodiment, the invention relates to a device comprising a composition for use in the prevention and/or treatment of the damages caused by retinal detachment in any of the embodiment above described.

In a specific embodiment, the device of the invention is a syringe; preferably a prefilled syringe.

### DEFINITIONS

In the present invention, the following terms have the following meanings:
“About” is used herein to mean approximately, roughly, around, or in the region of. The term “about” preceding a figure means plus or less 10% of the value of said figure. When the term “about” is used in conjunction with a numerical range, it modifies that range by extending the boundaries above and below the numerical values set forth by 10%.
“Injectable” means suitable for administration to the eye by means of injection. Injectability is also associated with an easier removal of an ophthalmic device after its presence in the eye is no longer necessary.
“Insulin sensitizers” refers to compounds that improves the sensitivity of cells to the metabolic effects of insulin. Example of insulin sensitizers comprises thiazolidinediones such as rosiglitazone, pioglitazone and MSDC-0602K and metformin.
“Intraocular treatment” refers to an ophthalmic treatment which comprise a step of intraocular administration of an ophthalmic substance or composition to an internal part of the eye of a subject such as, for example, eye cavity, anterior chamber (aqueous humour), trabecular meshwork, posterior chamber, uvea, lens, iris, ciliary body, vitreous cavity (vitreous humour), or retina. A specific intraocular treatment is intravitreal treatment, wherein the intraocular administration is into the vitreous humour. Another specific intraocular treatment is intracameral treatment, wherein the intraocular administration is into the eye cavities. Preferably, the intraocular administration is carried out by means of an intraocular injection.
“Ophthalmic substance” or “ophthalmic composition” refer to a substance or a composition intended to be administered to the eye of a subject and/or which is suitable to be administrated to the eye of a subject. Preferably, an ophthalmic substance or composition is indicated for treating an eye disease or condition.
“Ophthalmic treatment” refers to a treatment of an eye disease or condition in a subject in need thereof, which comprise a step of administration of an ophthalmic substance or composition to the eye of a subject.
“Pharmaceutically acceptable” used in conjunction with an ingredient of a composition, it is meant that the ingredients of a pharmaceutical composition are compatible with each other and not deleterious to the subject to which the pharmaceutical composition is administered.
“Pharmaceutically acceptable excipient” refers to an excipient or vehicle that does not produce an adverse, allergic or other untoward reaction when administered to a subject, preferably a human. It includes any and all solvents, dispersion media, coatings, antibacterial and antifungal agents, isotonic and absorption delaying agents, and the like. For human administration, preparations should meet sterility, pyrogenicity, general safety and purity standards as required by regulatory offices, such as, for example, Food and Drug Administration (FDA) Office or European Medicine Agency (EMA).
“Pharmaceutical composition” refers to an ophthalmic composition comprising at least a pharmaceutically active agent in association with at least a pharmaceutically acceptable excipient. A pharmaceutical composition is for therapeutic use, and relates to health. Especially, a pharmaceutical composition may be indicated for treating or preventing a disease. Preferably, a pharmaceutical composition is for use in the treatment of an eye disease or an eye condition.
“Retinal detachment” refers to a detachment between the neurosensory retina and the underlying retinal pigment epithelium.
“Risk of retinal detachment” refers to risk factors of rhegmatogenous retinal detachment which encompasses: posterior vitreous detachment, retinal holes, lattice degeneration, severe myopia, eye trauma, cataract surgery, risk factors for tractional retinal detachment encompass: diabetic retinopathy, sickling hemoglobinopathie, retinopathy of prematurity; risk factors for exudative retinal detachment encompass: uveitis (Vogt Koyanagi Harada disease, sympathetic ophthalmia, posterior scleritis), tumors (retinal capillary hemangioma, choroidal hemangioma, choroidal melanoma, choroidal metastasis, retinoblastoma), vascular (central serous retinopathy, Coats disease, eclampsia, malignant hypertension) or congenital (Morning glory syndrome, optic disc pit, coloboma, nanophthalmos).
“Subject” refers to a warm-blooded animal, preferably a mammal, more preferably a human. Preferably, the subject is a patient, i.e., a subject who is awaiting the receipt of, or who is receiving medical care, or who is/will be the object of a medical procedure. Preferably, the subject has an eye disease or an eye condition.
“Tamponade” refers to a fluid injectable in the vitreous to treat a retinal condition, e.g., retinal detachment.
“Therapeutic agent”, “active agent” and “pharmaceutically active agent” are synonyms and refer to a compound for therapeutic use, and relates to health. Especially, a therapeutic agent may be indicated for treating or preventing a disease or condition, as defined hereafter. Preferably, the disease treated by the therapeutic agent is an eye disease or an eye condition.
“Treating” or “treatment” or “alleviation” refers to both therapeutic treatment and prophylactic or preventative measures; wherein the object is to prevent or slow down (lessen) the targeted disease or condition in a subject in need thereof. Those in need of treatment include those already with the disease or condition as well as those prone to have the disorder or those in whom the disorder is to be prevented. A subject is successfully “treated” for a disease or condition if, after receiving a therapeutic amount of an substance or composition according to the present invention, the subject shows observable and/or measurable reduction in or absence of one or more of the following: reduction in the number of pathogenic cells; reduction in the percent of total cells that are pathogenic; relief to some extent, of one or more of the symptoms associated with the specific disease or condition; reduced morbidity and mortality; and/or improvement in quality of life issues. The above parameters for assessing successful treatment and improvement in the disease are readily measurable by routine procedures familiar to a physician. Preferably, the disease or condition is an eye disease or an eye condition, i.e., a pathologic disorder or a condition affecting the eye of a subject.
“Vitreous” refers to the vitreous or vitreal cavity of the eye, also called corpus vitreum.

### DETAILED DESCRIPTION

The invention deals with an insulin sensitizer for use in the prevention and/or treatment of the damages caused by retinal detachment.

According to the present invention, retinal detachment can be rhegmatogenous retinal detachment which is the most common form of retinal detachment where a full-thickness break in the neuroretina allows fluid from the vitreous cavity to gain access to the potential subretinal space, resulting in retinal separation. Other forms of retinal detachment are also part encompassed by the present invention, they refer to non-rhegmatogenous retinal detachment and include tractional (secondary to vascular fibrous membranes on the surface of the retina) and exudative (caused by leakage from retinal or choroidal vessels) retinal detachment.

As such, the present invention deals with a method of treatment and/or prevention of retinal detachment with an insulin sensitizer, a composition comprising an insulin sensitizer, more particularly this composition is a pharmaceutical composition or a medicament comprising an insulin sensitizer.

According to specific aspects of the invention, the damages caused by retinal detachment can be cone cell death and/or loss of vision.

According to another aspect, the invention deals with a composition for use in the prevention and/or treatment of the damages caused by retinal detachment, wherein said composition comprises at least one insulin sensitizer, wherein preferably the damages caused by retinal detachment is cone cell death and/or loss of vision.

As such, the present invention deals with a method of treatment and/or prevention of the damages caused by retinal detachment with an insulin sensitizer, a composition comprising an insulin sensitizer, more particularly this composition is a pharmaceutical composition or a medicament comprising an insulin sensitizer.

In a more specific aspect, the pharmaceutical composition and further comprises at least one pharmaceutically acceptable excipient.

Pharmaceutically acceptable excipients that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene- polyoxypropylene- block polymers, polyethylene glycol and wool fat.

According to another aspect of the invention, the composition further comprises at least one therapeutic agent; preferably an anti-inflammatory, anti-fibrotic, anesthetic, anti-proliferative, antiseptic, anti-infective, antioxidant and/or reactive oxygen species (ROS) scavenger agent.

According to another aspect of the invention, the composition further comprises at least one buffering agent, osmotic agent and/or colorant.

According to another aspect of the invention, the composition is an injectable solution; preferably an aqueous solution or a hydrogel solution.

Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent. Among the acceptable vehicles and solvents that may be employed are water, Ringer’s solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

According to a further aspect of the invention, the insulin sensitizer of the invention is chosen among direct PPAR agonists, selective PPAR gamma modulators and PPAR gamma-sparing compounds. Direct PPAR agonists can be chosen among the non-limitative list comprising thiazolidinediones such as rosigliatazone, pioglitazone, Edaglitazone, GW1929, troglitazone, Ciglitazone, or Troglitazone, selective PPAR gamma modulators can be chosen among the non-limitative list comprising

INT131, CMHX008, nTZDpa, S26948 or Pseudoginsenoside F11, PPAR gamma-sparing compounds can be chosen among the non-limitative list comprising ligand/modulator of the mTOT, agents target to the downstream effectors of PPAR gamma, stimulation of HSP/NOS (BGP-15), 11 beta-HSD1 inhibitor (INCB13739), AMPK activators, biguadine (metformin) or MSDC-0602K, chloroquines (CQs).

According to another aspect, the insulin sensitizer or the composition for use according to the present invention is to be administered to a subject at risk of retinal detachment, preferably before retinal detachment.

Examples of risks of developing rhegmatogenous retinal detachment include, but are not limited to retinal holes, lattice degeneration, severe myopia, eye trauma, cataract surgery and the like.

Examples of risks of developing tractional retinal detachment include, but are not limited to diabetic retinopathy, sickling hemoglobinopathies, retinopathy of prematurity and the like.

Examples of risks of developing exudative retinal detachment include, but are not limited to uveitis (Vogt Koyanagi Harada disease, sympathetic ophthalmia, posterior scleritis), tumors (retinal capillary hemangioma, choroidal hemangioma, choroidal melanoma, choroidal metastasis, retinoblastoma), vascular (central serous retinopathy, Coats disease, eclampsia, malignant hypertension), congenital (Morning glory syndrome, optic disc pit, coloboma, nanophthalmos) and the like.

According to a further aspect, the insulin sensitizer or the composition for use according to the invention is to be administered to a subject after initiation of retinal detachment.

According to a still further aspect, the insulin sensitizer or the composition for use according to the present invention is to be administered as long as there are functional cone photoreceptors.

According to a still further aspect, the insulin sensitizer or the composition for use according to the present invention is to be administrated by direct retinal, subretinal or intravitreal injection.

In one embodiment, the composition, pharmaceutical composition, or medicament of the invention is administered to the subject in need thereof at least once a day. For example, the composition, pharmaceutical composition, or medicament of the invention may be administered once a day, twice a day, or three times a day. In a preferred embodiment, the composition, pharmaceutical composition, or medicament of the invention is administered to the subject in need thereof once a day.

In another embodiment, the composition, pharmaceutical composition, or medicament of the invention is administered to the subject in need thereof at least once a week. For example, the composition, pharmaceutical composition, or medicament of the invention may be administered once a week, twice a week, three times a week, four times a week or up to seven times a week.

In another embodiment, the composition, pharmaceutical composition, or medicament of the invention is administered to the subject in need thereof once a month, two times a month, every two months, every two or three month, two times a year or once a year.

Another object of the present invention is a method for treating retinal detachment in a subject in need thereof, comprising administering to the subject an effective amount of the insulin sensitizer of the invention.

The present invention also relates to a kit comprising at least one insulin sensitizer, a pharmaceutical composition or a medicament according to the invention.

In one embodiment, the kit of the invention is used for treating (or for use in treating) retinal detachment.

In one embodiment, the kit of the invention further comprises means to administer the insulin sensitizer, the pharmaceutical composition or the medicament to a subject in need thereof.

In one embodiment, the kit of the invention further comprises instructions for the administration of the agent, the pharmaceutical composition or the medicament to said subj ect.

In another embodiment, the kit of the invention comprises two parts wherein the first part comprises the at least one insulin sensitizer, pharmaceutical composition or medicament according to the invention, and wherein the second part comprises an additional preventive and/or therapeutic agent. According to one embodiment, said additional preventive and/or therapeutic agent is another agent for treating retinal detachment.

In one embodiment, the components of the kit of parts of the invention may be administered separately, sequentially, simultaneously, concurrently or chronologically staggered.

In one embodiment, the part of the kit of part comprising the additional preventive and/or therapeutic agent is in a form adapted to the same administration route than the at least one insulin sensitizer, pharmaceutical composition or medicament of the invention. In another embodiment, the part of the kit of part comprising the additional preventive and/or therapeutic agent is in a form adapted to another administration route than the at least one insulin sensitizer, pharmaceutical composition or medicament of the invention.

According to another aspect, the invention deals with a device comprising a composition for use according to the present invention.

In more specific aspect, the device is a syringe; preferably a prefilled syringe.

### BRIEF DESCRIPTION OF THE DRAWINGS

FIGS. 1A-1H: are a combination of graphs showing that insulin is essential for cone survival in vitro and delays RD-induced cone loss in vivo. FIG. 1A and FIG. 1B represent CAR+ cones after 5 days of culture in high glucose DMEM (25 mM) with (FIG. 1A) or without (FIG. 1B) human insulin and/or insulin receptor inhibitor (HNMPA) or its vehicle (n = 5/group; FIG. 1A: one-way Anova *p = 0.0170 and $p = 0.0001 versus the high glucose culture; FIG. 1B: one-way Anova *p = 0.0005 and $p = 0.0001 versus the high glucose culture). FIG. 1C represents the quantification of subretinal IBA-1+ cells, FIG. 1D represents the quantification of PNA+ cones and FIG. 1E CAR+ cones in retinal flatmounts of C57BL6/J mice without RD and 7 days after RD with or without subretinal injection of insulin (n = 6-12/group;

FIG. 1C: one-way Anova *p < 0.0001 and $p < 0.0001 versus the control group;

FIG. 1D: one-way Anova *p < 0.0001 versus the control group, $p = 0.0018 versus the PBS group; FIG. 1C: one-way Anova *p < 0.0001 versus the control group, $p = 0.0002 versus the PBS group). FIG. 1F represents the quantification of subretinal IBA-1+ cells, FIG. 1G represents PNA+ cones and FIG. 1H represents tAR+ cones in retinal flatmounts of C57BL6/J mice without RD and 7 days after RD with or without subretinal injection of IGF-1 (n = 6-12/group; FIG. 1F: one-way Anova *p < 0.0001, $p < 0.0001 versus the control group; FIG. 1G: one-way Anova *p < 0.0001 versus the control group; FIG. 1H: one-way Anova *p < 0.0001 versus the control group) wherein PNA: peanut agglutinine; CAR: cone arrestin; IBA-1: ionized calcium-binding adapter molecule 1; IR: insulin receptor; DMSO: dimethyl sulfoxyde; RD: retinal detachment; PBS: phosphate buffered saline; IGF-1: insulin-like growth factor; DMEM: Dulbecco’s modified Eagle’s medium; HNMPA: hydroxy-2-naphthalenylmethylphosphonic acid. All values are reported as mean ± SEM.

FIGS. 2A-2F are a combination of graphs showing that the insulin sensitizers rosiglitazone and metformin prevent RD-induced cone loss. FIG. 2A represents the quantification of subretinal IBA-1+ cells, FIG. 2B represents the quantification of PNA+ cones and FIG. 2C represents the quantification of CAR+ cones in retinal flat mounts of C57BL6/J mice without RD and 7 days after RD and treatment with rosiglitazone or its vehicle (n = 6-12/group; FIG. 2A: one-way Anova *p < 0.0001 and $p < 0.0001 versus the control group; FIG. 2B: one-way Anova *p < 0.0001 versus the control group, $p = 0.0017 versus the DMSO group; FIG. 2C: one-way Anova *p < 0.0001 versus the control group, $p = 0.0029 versus the DMSO group).

FIG. 2D represents the quantification of subretinal IBA-1+ cells, FIG. 2E represents the quantification of PNA+ cones and FIG. 2F represents the quantification of CAR+ cones in retinal flat mounts of C57BL6/J mice without RD and 7 days after RD without or with subretinal injection of metformin (n = 6-12/group;

FIG. 2D: one-way Anova *p < 0.0001 versus the control group, $p < 0.0001 versus the PBS group; FIG. 2E: one-way Anova *p < 0.0001 versus the control group, $p = 0.0056 versus the PBS group; FIG. 2F: one-way Anova *p < 0.0001 versus the control group, $p = 0.0004 versus the PBS group). In FIGS. 2D-2F, RD means retinal detachment; DMSO: dimethyl sulfoxyde; RGZ: rosiglitazone; PNA: peanut agglutinine; CAR: cone arrestin; IBA-1: ionized calcium-binding adaptater molecule 1; PBS: phosphate buffered saline. All values are reported as mean ± SEM.

### EXAMPLES

The present invention is further illustrated by the following examples.

### Example 1: Material and Methods

### Patients

We conducted a nonrandomized clinical study at Nancy University Hospital from November 2017 to August 2018. Forty-one patients with primary RD requiring vitrectomy and 33 control patients undergoing vitrectomy for vitreomacular traction (VMT) or macular hole (MH) were included in this study. This study was approved by the regional Institutional Ethics Committee (Comite de Protection des Personnes CPP17-059/2017-A02195-48) and adhered to the Declaration of Helsinki. All patients gave their written consent before surgery.

Exclusion criteria were any history of vitreoretinal surgery on the eye studied, diabetic retinopathy or uveitis.

All patients underwent a detailed ophthalmologic examination before surgery, including best-corrected visual acuity (BCVA) measured with projected-light Snellen charts, axial length measurement using IOLMaster (Carl Zeiss Meditec, Dublin, CA), biomicroscopy with anterior segment evaluation, fundus and careful peripheral retina examination. For the RD group, an Amsler-Dubois scheme was systematically established for each patient, specifying the extent of the RD, number, type and location of retinal breaks, existence of vitreous hemorrhage and preoperative proliferative vitreoretinopathy (PVR) grading according to Machemer et al (Machemer et al., 1991). For the control group, all patients underwent a spectral-domain optical coherence tomography (Heidelberg Spectralis-OCT, Heidelberg Engineering, Heidelberg, Germany) imaging to measure either the size of attachment area in case of VMT or the size of macular hole using the calipers provided by the manufacturer’s software.

All patients underwent a three-port 23- or 25-gauge pars plana vitrectomy. At the beginning of vitrectomy, air perfusion was set to open and undiluted vitreous fluid samples (1 mL) were collected from each eye with 3 mL syringe. Samples were sent to the Biological Resource Center (Centre de Ressources biologiques, Nancy, France) within 30 min, cooled on ice and transferred into microfuge tubes. Each sample was centrifuged at 10000 g for 5 min and the supernatant was then collected and frozen at -80° C. before analysis.

### Glucose Assay

Glucose concentration was measured using Glucose Assay Kit (Abcam, Cambridge, UK). Briefly, vitreous samples were thawed and 8 µL of undiluted supernatant were used for glucose titration in accordance with the manufacturer’s instructions. All samples were tested in simplicate.

### Mouse Model of RD

Wild-type (C57BL/6J) mice were purchased from Janvier Labs at the age of 8 weeks. Mice were housed in the animal facility under specific pathogen-free condition, in a 12 h/12 h light/dark (100-500 lux) cycle with water and normal diet food available ad libitum. All experimental protocols and procedures were approved by the local animal care ethics committee (N°APAFIS#5201-20160427103344).

RD was induced with a previously described method (Nakazawa et al., 2006, 2007a, 2011). Briefly, mice were anesthetized with an intraperitoneal injection of xylazine hydrochloride (10 mg/kg) and ketamine hydrochloride (100 mg/kg) and pupils were dilated with topical phenylephrine (5%) and tropicamide (0.5%). A 30-gauge needle was first used to create two sclerotomies 1.5 mm posterior to the limbus. A glass needle (with a 80-gauge manually beveled tip) connected to a Hamilton syringe filled with diluted sodium hyaluronate (Healon GV®, Alcon) was then introduced into the vitreous cavity through one of the sclerotomy. The tip of the needle was finally inserted into the subretinal space through a peripheral retinotomy and 4 µl of diluted sodium hyaluronate containing or not recombinant human TSP-1 (100 µg/ml, Biotechne), human insulin (2 IU/ml, Umuline NPH), recombinant IGF-1 (200 ng/ml, Biotechne) or metformin (50 mg/ml), was gently injected, detaching approximately two-third of the retina from the underlying RPE.

For treatment with rosiglitazone, mice received intraperitoneal injections of 10 mg/kg rosiglitazone or vehicle (5% DMSO) 3 days before and 4 to 7 days after RD induction.

Eyes with subretinal hemorrhage were excluded from analysis. Mice were sacrificed from 1 to 10 days following RD, according to the experiment.

### Retinal Explants Culture

C57BL/6J retina were prepared and placed on polycarbonate filters floating on Dulbecco’s modified Eagle’s medium (DMEM, Thermo Fisher Scientific) with the photoreceptors facing down. For the experiment, retinal explants were cultured in high glucose (25 mM) DMEM alone or supplemented with human insulin (Umuline NPH), human insulin and insulin receptor inhibitor (Hydroxy-2-naphthalenylmethylphosphonic acid (HNMPA), Abcam) or vehicle (DMSO) at 37° C. As insulin has been demonstrated to be quite unstable in media containing cysteine (Laskowski et al., 2016), we used supraphysiological doses of insulin (5 mIU/ml) in our model. For this reason, HNMPA was used at a concentration 10-fold higher than the IC50 (1 mM). Each culture medium was renewed every 36 hours and after 5 days, the retinal explants were carefully removed. Immunohistochemistry and cones quantification were then performed as described for retina below.

### Isolation of Retinal Immune Cells and Flow Cytometry Analyses

Retinas were dissected out and homogenized in 500 µL of PBS with liberase TL at 0.8 wunsch/mL (Sigma-Aldrich) for 30 min at 37° C. and 5% CO2. The retinal homogenate was washed with PBS and the pellet containing the immune cells was re-suspended in 100 µL PBS containing 1 µL of Viobility 405/520 Fixable Dye (Miltenyi). Cells were washed and labelled with 50 µL of primary antibodies mix: anti-CD45-VioBlue (REA737), anti-MHCII-FITC (REA813), anti-CD11b-PE (REA592), anti-Ly-6C-PE-Vio770 (REA796), anti-CD3-APC (REA641) and anti-Ly-6g-APC-Vio770 (REA526) (Miltenyi). After labeling, cells were fixed in 4% paraformaldehyde. For compensation settings, single-stained cellular controls with corresponding antibodies were used. Fluorescence intensities were measured using a MACSQuant analyzer (Miltenyi) and data were analyzed using the FlowJo Software.

### Immunohistochemistry of Retinal Flat Mounts

Eyes were enucleated, fixed in 4% paraformaldehyde for 1 hour at room temperature and sectioned at the limbus; the cornea and lens were discarded. The retinas were peeled from the RPE/choroid/sclera and incubated overnight at 4° C. in PBS-1% triton with the following primary antibodies: peanut agglutinin Alexa fluor® 594 (Thermo Fisher Scientific; 1/100), rabbit polyclonal anti-human cone arrestin antibody (LUMIF-hCAR; 1:10000) and goat polyclonal anti-IBA1 (1/100). After few washes, the retinas were incubated for 2 hours at room temperature with appropriate Alexa Fluor® conjugated secondary antibodies (Thermo Fisher Scientific; 1:500) in PBS-1% triton and nuclei were counterstained with Hoechst (1:1000, Sigma Aldrich). The retinas were flat mounted and viewed with a fluorescence microscope (DM5500, Leica). Images centered on the area with the lowest number of PNA+ cone arrestin+ cells were captured with a confocal laser-scanning microscope (FV1000, Olympus) using a 40X lens. Each cell population was manually counted in a masked fashion. IBA-1+ cells were quantified on flat mounts on the outer segment side of the detached retina while PNA+ cone arrestin+ cells were counted on confocal microscopy Z-stacks using ImageJ software.

### Immunohistochemistry And Tunel Staining Ofretinal Cross-Sections

Eyes were enucleated and fixed for one hour in 4% formaldehyde solution at 4° C. Cornea and lens were removed and eyecups were cryoprotected overnight in 30% sucrose solution in PBS (Sigma-Aldrich) at 4° C. The next day, samples were embedded in OCT (Neg 50, Richard-Allan Scientific), frozen in liquid nitrogen, and stored at -20° C. Sections (12 µm thick) were cut on a Microm Cryostat (Microm Microtech, France) at -20° C. and mounted on SuperFrost@Plus slides (Menzel-Gläser, Germany).

For immunohistochemistry, sections were incubated overnight at 4° C. with peanut agglutinin Alexa fluor 488 (Thermo Fisher Scientific; 1/100) and rabbit polyclonal anti-human cone arrestin antibody (LUMIF-hCAR; 1:10000) in PBS-1% BSA. Sections were then incubated for 1 hour at room temperature with a secondary antibody produced in donkey (Alexa Fluor® 647 nm, Thermo Fisher Scientific; 1:500) and nuclei were counterstained with Hoechst (1:1000, Sigma Aldrich). For TUNEL staining (In Situ Cell Death Detection Kit, Roche Diagnostics, Mannheim,Germany), after washing with PBS, sections were incubated for 1 hour at 37° C. with the reaction mixture and the reaction was stopped by washing with PBS. The slides were then mounted and observed with a fluorescence microscope (DM5500, Leica).

### Reverse Transcription and Real-Time Quantitative Polymerase Chain Reaction

Total RNA was extracted from mouse retina with the Nucleospin RNAII extraction kit according to the manufacturer’s protocol (Macherey Nagel). Single-stranded cDNA was synthetized from total mRNA (pretreated with DNase) using oligo-dT as primer and superscript II reverse transcriptase (Thermo Fisher Scientific). Subsequent RT-PCR was performed using cDNA, PowerSYBR Green PCR Master Mix (Applied Biosystems) and primers (IDT technology) available upon request. qPCR was performed using StepOne Plus real-time PCR systems (Applied Biosystems) with the following profile: 45 cycles of 15 s at 95° C., 45 s at 60° C. Results were normalized using house-keeping gene RPS26.

### Statistical Analysis

Graph Pad Prism 7 (GraphPad Software) was used for data analysis and graphic representation. All values are reported as mean ± SEM. Statistical analyses were performed by one-way Anova analysis of variance, Student t-test or Mann-Whitney U test for comparison among means depending on the experimental design. The p values are indicated in the figure legends.

### Example 2: Insulin Is Essential for Cone Survival in Vitro and Delays RD-Induced Cone Loss In Vivo

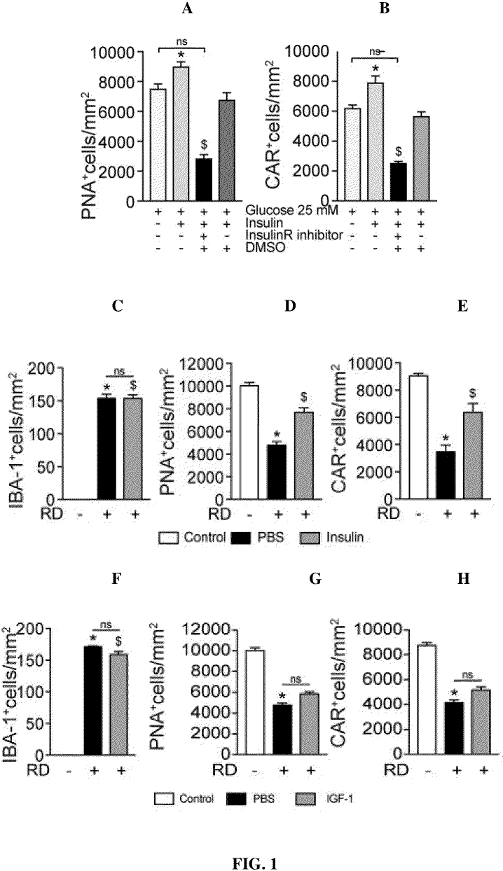

To investigate whether diminished insulin signaling could contribute to RD-associated cone loss in adult retinas, we first examined the effect of insulin on 5-day retinal explants with or without human insulin and/or the specific insulin receptor inhibitor HNMPA (FIG. 1A). Quantification of PNA+CAR+ cones showed that cone survival was significantly increased in the presence of insulin in the medium culture compared to the control condition (FIGS. 1A and 1B). The addition of an insulin receptor inhibitor HNMPA that blocks insulin receptor autophosphorylation, but not insulin growth factor 1 (IGF-1) receptor activation (Wang et al., 2014), resulted in a severe loss of PNA+CAR+ cones, not observed with its vehicle (DMSO) (FIGS. 1A and 1B). Our results indicate that insulin signaling promotes cone survival in retinal explants.

Next, we induced RD in vivo with subretinal injection of diluted sodium hyaluronate containing (or not) human insulin (2 IU/ml). Quantification on immuno-stained retinal flat-mounts at day 7 revealed that insulin treatment did not alter the numbers of subretinal IBA1+ MPs, but very significantly increased the number of PNA+CAR+ cones compared to PBS controls (FIGS. 1C-E). Comparatively, addition of IGF-1 to the detachment inducing gel (at a concentration 100-fold higher than IGF-1s ED50, which has been shown to reverse hypoalgesia in diabetic mice (Chu et al., 2008)) had no effect on the numbers of IBA1+ MPs or PNA+CAR+ cones, quantified on day 7 immuno-stained retinas (FIGS. 1F-H).

Taken together, our results showed that insulin and insulin receptor signaling were essential for cone survival ex vivo of adult retinas and that insulin treatment very significantly inhibited RD-induced cone loss despite the unchanged MPs infiltration in vivo. This effect was not due to insulin-induced IGF-1R signaling, which can activate anti-apoptotic IGF-1 receptor signaling (Kim and Accili, 2002; Siddle et al., 2001), as IGF-1 had no comparable effect.

### Example 3: The Insulin Sensitizers Rosiglitazone and Metformin Prevent RD-Induced Cone Loss

We next examined whether rosiglitazone and metformin could prevent cone loss in our mouse model of RD.

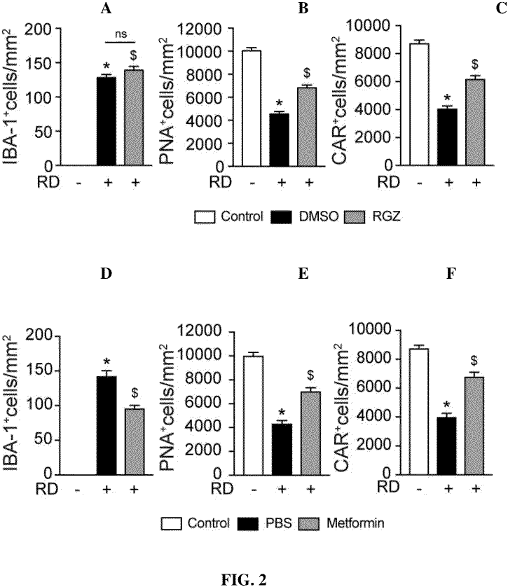

Mice received daily intraperitoneal injections of rosiglitazone or vehicle (DMSO 5%) 3 days before and throughout the 7 days of RD. Quantification of IBA1-, PNA-, CAR triple-stained retinal flat mounts at day 7 showed that rosiglitazone had no effect on the number of infiltration of subretinal IBA-1+ MPs (FIG. 2A), but very significantly inhibited PNA+CAR+ cone loss at day 7 of RD (FIGS. 2B and C). Interestingly, subretinal injection of metformin significantly decreased subretinal MPs accumulation and increased cone survival in detached retinas compared with PBS controls (FIGS. 2D-F).

In summary, our results show that the well-established insulin sensitizers rosiglitazone and metformin significantly curb cone loss in RD. The fact that we observed increased cone survival under rosiglitazone- and insulin- treatment in the absence of an anti-inflammatory effect strongly suggests that restored insulin signaling was the likely mode of action.

1-15. (canceled)

16. A method for preventing and/or treating damage caused by retinal detachment, wherein said method comprises administering to a subject an insulin sensitizer or a composition comprising at least one insulin sensitizer.

17. The method according to claim 16, wherein damage caused by retinal detachment is cone cell death.

18. The method according to claim 16, wherein damage caused by retinal detachment is loss of vision.

19. The method according to claim 16, wherein the composition is a pharmaceutical composition and further comprises at least one pharmaceutically acceptable excipient.

20. The method according to claim 16, wherein the composition further comprises at least one therapeutic agent.

21. The method according to claim 20, wherein the at least one therapeutic agent is selected from de group consisting of an anti-inflammatory, an anti-fibrotic, an anesthetic, an anti-proliferative, an antiseptic, an anti-infective, an antioxidant, a reactive oxygen species (ROS) scavenger agent, and combination thereof.

22. The method according to claim 16, wherein the composition further comprises at least one buffering agent, osmotic agent and/or colorant.

23. The method according to claim 16, wherein the composition is an injectable solution.

24. The method according to claim 23, wherein the injectable solution is an aqueous solution or a hydrogel solution.

25. The method according to claim 16, wherein the insulin sensitizer is selected from de group consisting of a direct PPAR agonist, a selective PPAR gamma modulator and a PPAR gamma-sparing compound.

26. The method according to claim 25, wherein the direct PPAR agonist is a thiazolidinedione selected from the group consisting of rosigliatazone, pioglitazone, Edaglitazone, GW1929, troglitazone, Ciglitazone, and Troglitazone; the selective PPAR gamma modulators is selected from the group consisting of INT131, CMHX008, nTZDpa, S26948 and Pseudoginsenoside F11; and the PPAR gamma-sparing compounds is selected from the group consisting of a ligand/modulator of the mTOT, agents target to the downstream effectors of PPAR gamma, stimulation of HSP/NOS (BGP-15), a 11 beta-HSD1 inhibitor (INCB 13739), an AMPK activator, biguadine (metformin) or MSDC-0602K, and a chloroquine (CQs).

27. The method according to claim 16, wherein the insulin sensitizer or the composition is to be administered to a subject at risk of retinal detachment.

28. The method according to claim 16, wherein the insulin sensitizer or the composition is to be administered to a subject before retinal detachment.

29. The method according to claim 16, wherein the insulin sensitizer or the composition is to be administered to a subject after initiation of retinal detachment.

30. The method according to claim 16, wherein the insulin sensitizer or the composition is to be administered as long as there are functional cone photoreceptors.

31. The method according to claim 16, wherein said insulin sensitizer or composition is to be administrated by direct retinal, subretinal or intravitreal injection.

32. A device comprising a composition according to claim 16.

33. A device according to claim 32, wherein the device is a syringe or a prefilled syringe.
